(19) United States
(12) Patent Application Publication
(10) Pub. No.: US 2022/0409840 A1
(43) Pub. Date: Dec. 29, 2022

(54) NASAL CANNULA

(71) Applicant: Vayu Global Health Innovations, LLC, Medford, MA (US)

(72) Inventors: Thomas F. Burke, Medford, MA (US); Russell T. Rapaport, Newton, MA (US); Ellie K. Ng, Waltham, MA (US); Michelle Dundek, Malden, MA (US)

(21) Appl. No.: 17/808,150

(22) Filed: Jun. 22, 2022

Related U.S. Application Data
(60) Provisional application No. 63/214,510, filed on Jun. 24, 2021.

(51) Int. Cl. A61M 16/06; A61M 16/08

(52) U.S. Cl. CPC A61M 16/0666; A61M 16/0833

(57) ABSTRACT

A nasal cannula includes a first tube, a second tube, a nasal connector tube, and a flow splitter. The nasal tube has first and second ends, the first end connected to a distal end of the first tube, the second end connected to a distal end of the second tube. First and second nasal prongs extend from and are fluidly coupled to the nasal connector tube. The flow splitter has an input and first and second outputs. The first output of the flow splitter is connected to a proximal end of the first tube. The second output of the flow splitter is connected to a proximal end of the second tube.

### CROSS-REFERENCE TO RELATED APPLICATIONS

This application claims priority to U.S. Provisional Application No. 63/214,510, titled “Nasal Cannula,” filed on Jun. 24, 2021, which is hereby incorporated by reference.

### TECHNICAL FIELD

This application relates generally to nasal cannulas.

### BACKGROUND

There are nearly 2 million cases of hypoxemic pneumonia in children under the age of 5 annually. Hypoxemia, or low blood oxygen concentration, is also a common complication of some of the most prevalent causes of newborn mortality, including birth asphyxia, sepsis, and low birth weight. Provision of supplemental oxygen can be a life-saving treatment for hypoxemic children. The supplemental oxygen is delivered to the patient through a nasal cannula.

### SUMMARY

Example embodiments described herein have innovative features, no single one of which is indispensable or solely responsible for their desirable attributes. The following description and drawings set forth certain illustrative implementations of the disclosure in detail, which are indicative of several exemplary ways in which the various principles of the disclosure may be carried out. The illustrative examples, however, are not exhaustive of the many possible embodiments of the disclosure. Without limiting the scope of the claims, some of the advantageous features will now be summarized. Other objects, advantages and novel features of the disclosure will be set forth in the following detailed description of the disclosure when considered in conjunction with the drawings, which are intended to illustrate, not limit, the invention.

An aspect of the invention is directed to a nasal cannula comprising: a first tube; a second tube; a nasal connector tube having first and second ends, the first end connected to a distal end of the first tube, the second end connected to a distal end of the second tube; first and second nasal prongs extending from and fluidly coupled to the nasal connector tube; and a Y-joint having an input and first and second outputs, the first output connected to a proximal end of the first tube, the second output connected to a proximal end of the second tube.

In one or more embodiments, the nasal cannula further comprises a collar having a channel through which the first and second tubes are inserted. In one or more embodiments, a loop is formed by a distal section of the first tube, a distal section of the second tube, the collar, and the nasal connector tube, the distal section of the first tube extends from the collar to the first end of the nasal connector tube, and the distal section of the second tube extends from the collar to the second end of the nasal connector tube. In one or more embodiments, the collar is configured to slidably engage the first and second tubes whereby a size of the loop is adjustable. In one or more embodiments, the first and second nasal prongs are curved to conform to a shape of a patient's nostrils. In one or more embodiments, when the first and second ends of the nasal connector tube lie approximately in a plane, the first and second nasal prongs extend from the connector tube at approximately a 45° angle with respect to the plane.

In one or more embodiments, the Y-joint comprises: a proximal tube; a first distal tube; and a second distal tube, wherein the proximal tube is fluidly coupled to the first and second distal tubes. In one or more embodiments, a proximal connector is attached to a proximal end of the proximal tube. In one or more embodiments, the proximal connector has a flared distal end. In one or more embodiments, the first and second distal tubes extend along a respective central axis, the Y-joint has an axis of symmetry, and the respective central axis and the axis of symmetry form an angle having a range of about 30° to about 60°. In one or more embodiments, the proximal tube extends along the axis of symmetry. In one or more embodiments, the angle is about 45°.

In one or more embodiments, a respective fillet is defined between (a) the first and second distal tubes and (b) the proximal tube. In one or more embodiments, the respective fillet has a filet radius having a range of about 0.8 mm to about 1.2 mm. In one or more embodiments, the filet radius is about 1 mm.

In one or more embodiments, the proximal connector is a second proximal connector, and the Y-joint further comprises a first proximal connector, the second proximal connector between the first proximal connector and the proximal tube. In one or more embodiments, each of the first and second proximal connectors has a flared distal end. In one or more embodiments, the first proximal connector is sized to be mechanically coupled to a first source gas tube having a first internal diameter, the second proximal connector is sized to be mechanically coupled to a second source gas tube having a second internal diameter, and the second internal diameter is greater than the first internal diameter.

Another aspect of the invention is directed to a system comprising: a gas source having a gas source output; and a nasal cannula comprising: a first tube; a second tube; a nasal connector tube having first and second ends, the first end connected to a distal end of the first tube, the second end connected to a distal end of the second tube; first and second nasal prongs extending from and fluidly coupled to the nasal connector tube; and a Y-joint having an input and first and second outputs, the first output connected to a proximal end of the first tube, the second output connected to a proximal end of the second tube, the input coupled to the gas source output.

### BRIEF DESCRIPTION OF THE DRAWINGS

Fora fuller understanding of the nature and advantages of the concepts disclosed herein, reference is made to the detailed description of preferred embodiments and the accompanying drawings.

FIG. 1 is a top view of a nasal cannula according to an embodiment.

FIG. 2 is a top perspective view of the nasal cannula illustrated in FIG. 1.

FIG. 3 is another top view of the nasal cannula illustrated in FIG. 1.

FIG. 4 is a rear perspective view of the nasal cannula illustrated in FIG. 1.

FIGS. 5 and 6 are side perspective views of the nasal cannula illustrated in FIG. 1.

FIG. 7 is top perspective view of a nasal cannula according to another embodiment.

FIG. 8 is a top view of a Y-joint according to an embodiment.

FIG. 9 is a top view of a Y-joint according to an embodiment.

FIG. 10 is a perspective view of a system according to an embodiment.

### DETAILED DESCRIPTION

A nasal cannula includes a flow splitter, first and second tubes, and a nasal connector tube. The flow splitter is configured to be fluidly coupled to a source of air and/or enriched oxygen. The flow splitter receives gas from the air and/or enriched oxygen source and splits the gas into first and second output streams which output the flow splitter through first and second flow splitter output tubes, respectively. The flow splitter can be a Y-splitter. The first and second tubes fluidly couple the nasal connector tube to the first and second flow splitter output tubes. The nasal connector tube includes first and second nasal tubes that can deliver the air and/or enriched oxygen to a patient's nostrils.

The first and second tubes can pass through a channel in an optional collar that can slide over the first and second tubes. The position of the collar along the first and second tubes is slidably adjustable. A first loop is formed by the nasal connector tube, the first and second tubes, and the collar. A second loop is formed by the collar, the first and second tubes, and the flow splitter. A change in position of the collar with respect to the first and second tubes changes the size of the first and second loops.

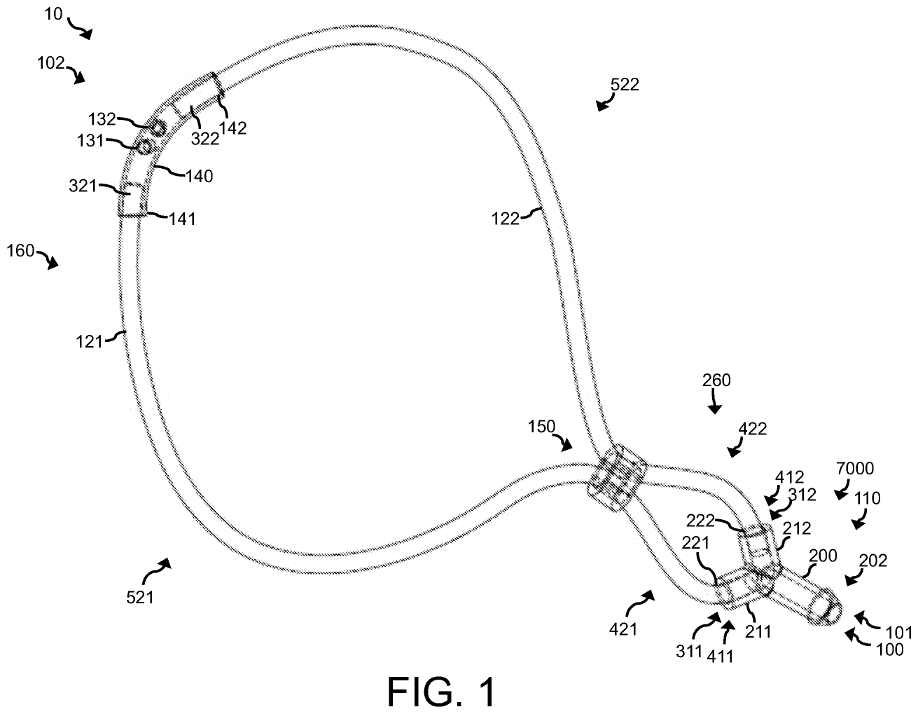

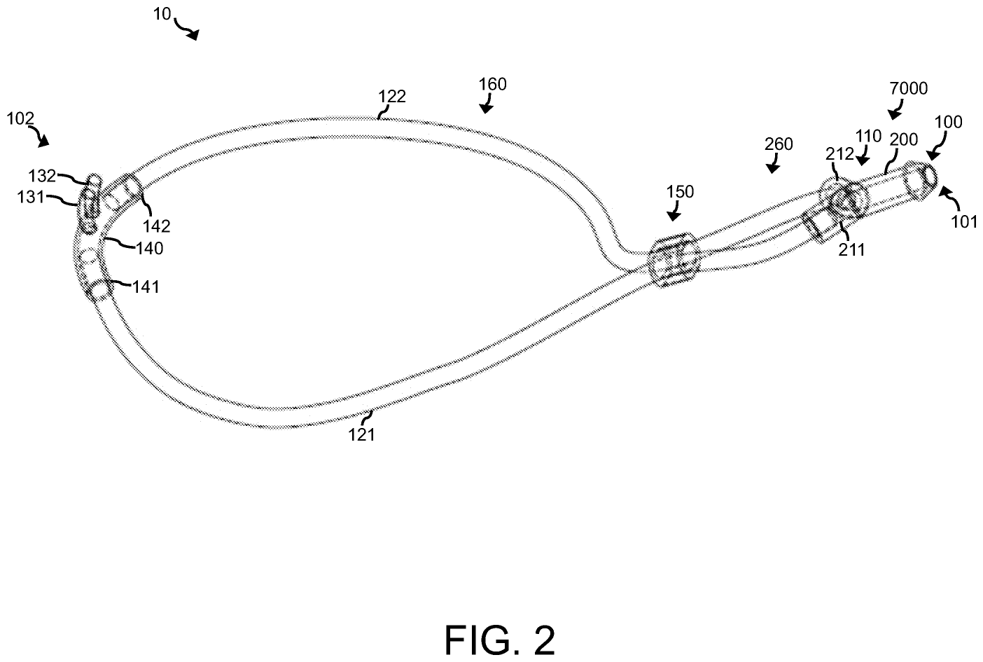

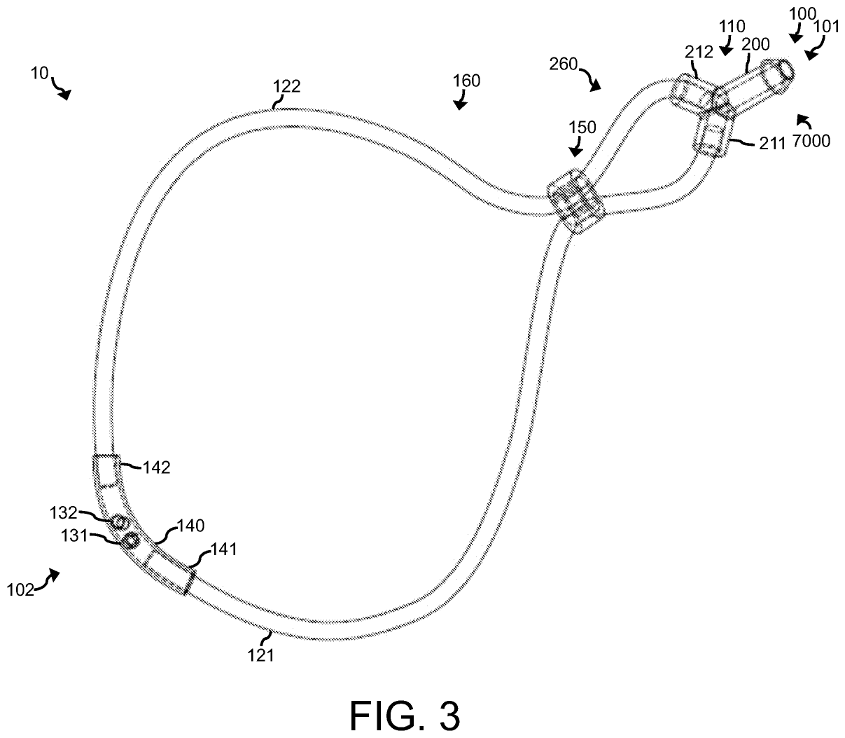

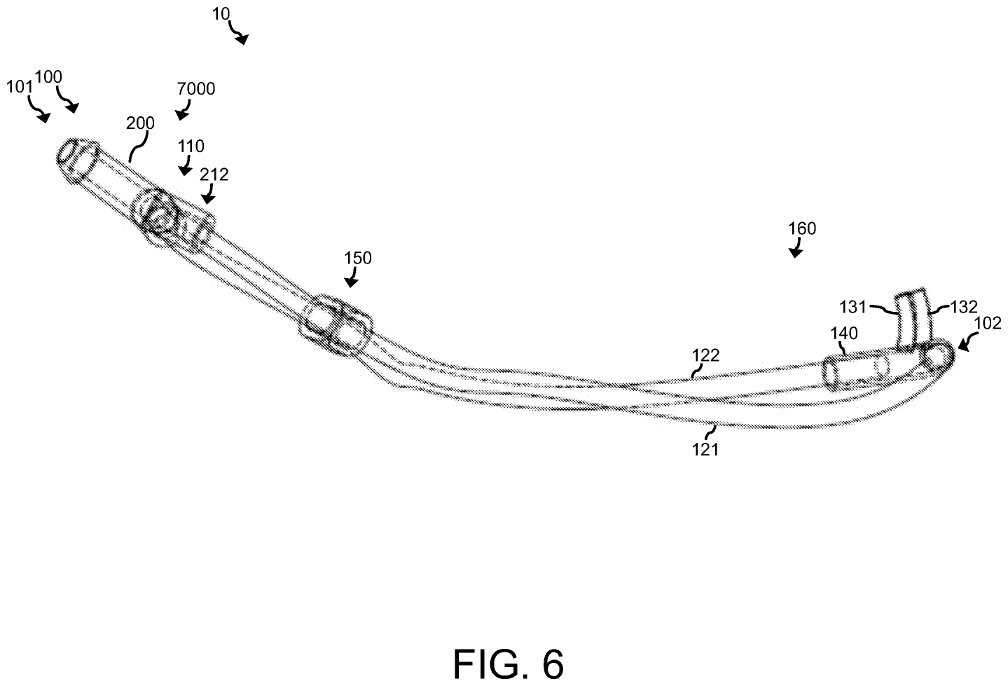

FIGS. 1-6 illustrate various views of a nasal cannula 10 according to an embodiment. The nasal cannula 10 includes a plurality of tubes through which air and/or oxygen can flow from an air and/or oxygen source to a patient's nostrils (e.g., nares).

The nasal cannula 10 includes a flow splitter 110, first and second tubes 121, 122, a nasal connector tube 140, and an optional collar 150. As illustrated, the flow splitter 110 is located at a proximal end 101 of the nasal cannula 10, and the nasal connector tube 140 is located at a distal end 102 of the nasal cannula 10. The flow splitter 110, the first and second tubes 121, 122, and the nasal connector tube 140 are fluidly coupled to each other.

The flow splitter 110 includes an input or connection end 100 that is configured to be releasably coupled to a source of air and/or oxygen. The connection end 100 can be friction and/or interference fit to a port to receive the air and/or oxygen. An example of an air and/or oxygen source is a blended low-flow oxygen delivery system, which can include, for example, the blended low-flow oxygen delivery system disclosed in U.S. Patent Application Publication No. 2018/0333555, titled “Adjustable Ambient Air-Oxygen Blender,” published on Nov. 22, 2018, which is hereby incorporated by reference.

The flow splitter 110 can comprise a Y-joint (e.g., as illustrated), a T-joint, or another flow splitter (e.g., a 3-way flow splitter). The flow splitter 110 includes a proximal tube 200 and first and second distal tubes 211, 212. The connection end 100 is located at a proximal end 202 of and/or attached to the proximal end 202 of the proximal tube 200. The first and second distal tubes 211, 212 are fluidly coupled and connected to the proximal tube 200. In operation, air and/or oxygen enters the flow splitter 110 through the connection end 100, passes through the proximal tube 200, the first and second distal tubes 211, 212, and first and second outputs 311, 312, respectively. The first and second outputs 311, 312 are located at the distal end 411, 412 of the first and second distal tubes 211, 212, respectively.

The first and second tubes 121, 122 are connected to and fluidly couple the first and second distal tubes 211, 212, respectively, and the nasal connector tube 140. For example, a proximal end 221 of the first tube 121 is connected to and/or inserted into the distal end 411 of the first distal tube 211 to receive the gas (e.g., air and/or oxygen) therefrom. Alternatively, the distal end 411 of the first distal tube 211 can be connected to and/or inserted into the proximal end 221 of the first tube 121. A proximal end 222 of the second tube 122 is connected to and/or inserted into the distal end 412 of the second distal tube 212 to receive the gas (e.g., air and/or oxygen) therefrom. Alternatively, the distal end 412 of the second distal tube 212 can be connected to and/or inserted into the proximal end 222 of the second tube 122.

A distal end 321 of the first tube 121 can be connected to and/or inserted into a first end 141 of the nasal connector tube 140 to provide the gas (e.g., air and/or oxygen) thereto. Alternatively, the first end 141 of the nasal connector tube 140 can be connected to and/or inserted into the distal end 321 of the first tube 121. A distal end 322 of the second tube 122 can be connected to and/or inserted into a second end 142 of the nasal connector tube 140 to provide the gas (e.g., air and/or oxygen) thereto. Alternatively, the second end 142 of the nasal connector tube 140 can be connected to and/or inserted into the distal end 322 of the second tube 122.

The first and second ends 141, 142 of the nasal connector tube 140 can be permanently attached (e.g., bonded such as with a solvent or adhesive) or releasably attached (e.g., by friction and/or interference fit) to the distal ends 321, 322 of the first and second tubes 121, 122, respectively. Additionally or alternatively, the distal ends 321, 322 of the first and second tubes 121, 122 can be permanently or releasably attached (e.g., by friction and/or interference fit) to the first and second ends 141, 142, respectively, of the nasal connector tube 140.

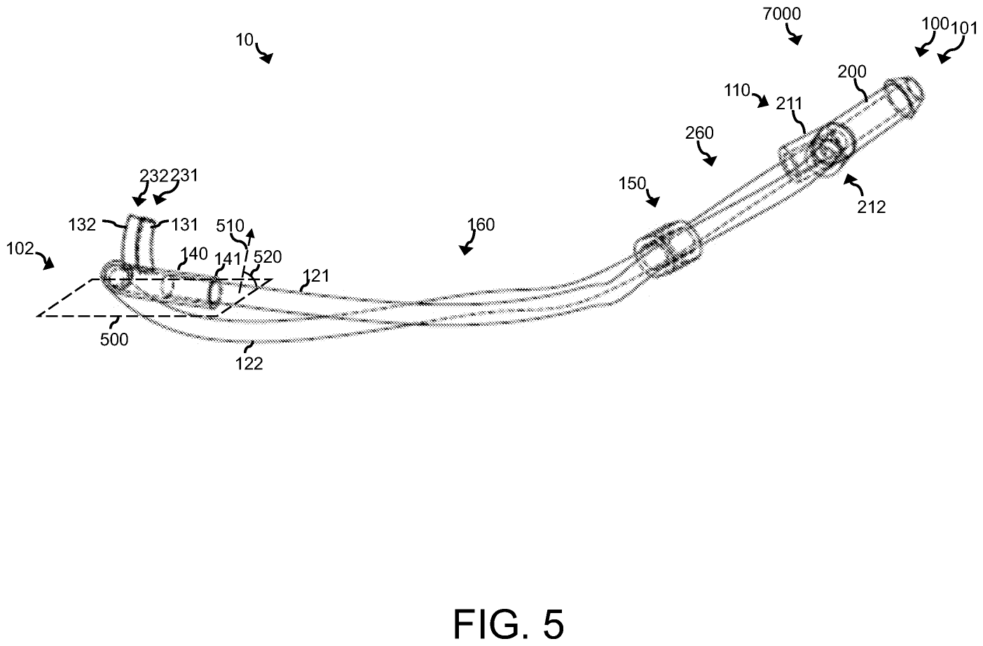

The nasal connector tube 140 is fluidly coupled to first and second nasal prongs or tubes 131, 132 that extend out of the nasal connector tube 140. The nasal tubes 131, 132 can be integrally connected to the nasal connector tube 140. Alternatively, the nasal tubes 131, 132 can be separate components that can be releasably attached to the nasal connector tube 140. When the nasal connector tube 140 (e.g., the first and second ends 141, 142 of the nasal connector tube 140) lies in a plane 500 (FIG. 5) or approximately in (e.g., within 25° of) the plane 500, the nasal tubes 131, 132 can extend along or parallel to an axis 510 that forms an angle 520 with respect to the plane 500. The angle 520 can have a range of about 0° to about 90°, including about 15°, about 30°, about 45°, about 60°, about 75°, and any value or range between any two of the foregoing angles. Additionally or alternatively, the nasal tubes 131, 132 can be curved to match or approximately match the curvature of the patient's nostrils. The nasal tubes 131, 132 can have open distal ends 231, 232 to release the gas (e.g., air and/or oxygen) into the patient's nostrils. Additionally or alternatively, one or more holes can be defined in the nasal tubes 131, 132 to release the gas into patient's nostrils. As used herein, “about” and “approximately” mean plus or minus 10% of the relevant value.

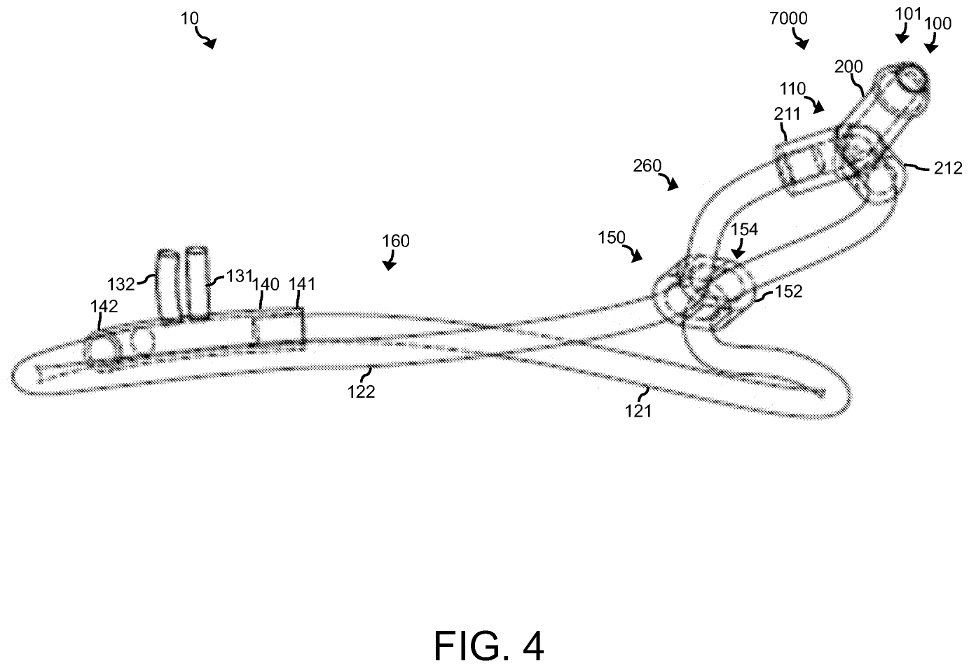

The optional collar or ring 150 is slidably adjustably attached to the first and second tubes 121, 122 between the flow splitter 110 and the nasal connector tube 140. The collar 150 has a body 152 that defines a channel 154 through which the first and second tubes 121, 122 are inserted, as illustrated in FIG. 4. In another embodiment, the body 152 can define a separate channel for each tube 121, 122. The collar 150 divides the first and second tubes 121, 122 into proximal sections 421, 422 and distal sections 521, 522, respectively, as illustrated in FIG. 1. A first loop 160 is defined by the distal sections 521, 522 of the first and second tubes 121, 122, respectively, the nasal connector tube 140, and the collar 150. A second loop 260 is defined by the proximal sections 421, 422 of the first and second tubes 121, 122, respectively, the collar 150, and the flow splitter 110.

The size or diameter of the loops 160, 260 can be changed by moving the collar 150 with respect to the first and second tubes 121, 122. For example, the size/diameter of the first loop 160 can be increased by pushing the collar 150 towards the flow splitter 110 while holding the first and second tubes 121, 122 such that the collar 150 slides over the first and second tubes 121, 122 towards the flow splitter 110. Additionally or alternatively, the size/diameter of the first loop 160 can be increased by pushing or pulling the first and second tubes 121, 122 towards the nasal connector tube 140 while holding the collar 150 such that the first and second tubes 121, 122 slide through the channel 154 towards the nasal connector tube 140. An increase in size/diameter of the first loop 160 causes a corresponding decrease in size/diameter of the second loop 260.

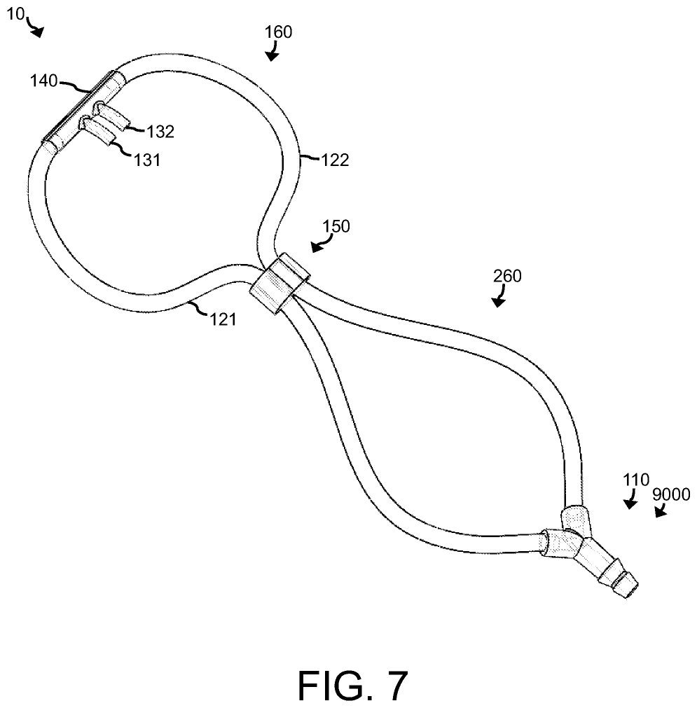

In another example, the size/diameter of the first loop 160 can be decreased by pushing the collar 150 towards the nasal connector tube 140 while holding the first and second tubes 121, 122 such that the collar 150 slides over the first and second tubes 121, 122 towards the nasal connector tube 140. Additionally or alternatively, the size/diameter of the first loop 160 can be decreased by pushing the first and second tubes 121, 122 towards the flow splitter 110 while holding the collar 150 such that the first and second tubes 121, 122 slide through the channel 154 towards the flow splitter 110. FIG. 7 is an example illustration of the nasal cannula 10 where the size/diameter of the first loop 160 is smaller compared to the size/diameter of the second loop 260 illustrated in FIGS. 1-6. A decrease in size/diameter of the first loop 160 causes a corresponding increase in size/diameter of the second loop 260.

In general, the tubing (e.g., first and second tubes 121, 122, nasal connector tube 140, and/or other tubing) of the nasal cannula 10 can be flexible to allow the nasal cannula 10 to be adjusted in a variety of configurations to suit the needs of the provider and/or patient. In addition or in the alternative, the tubing (e.g., first and second tubes 121, 122, nasal connector tube 140, and/or other tubing) can be crush-resistant. The flow splitter 110 is preferably rigid and/or crush-resistant.

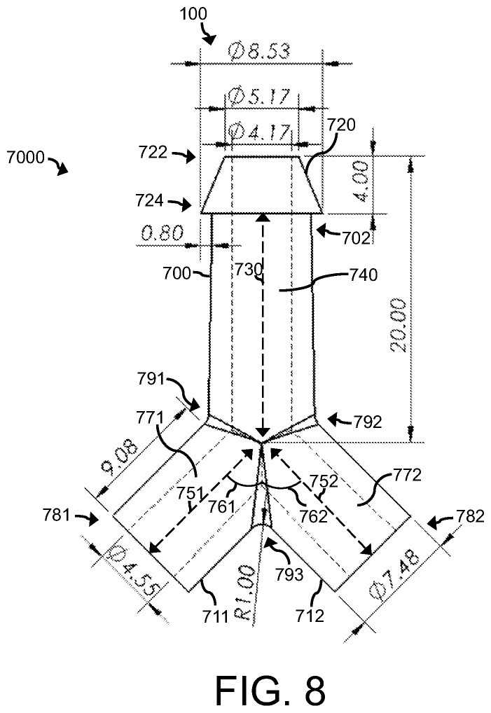

FIG. 8 is a top view of a Y-joint 7000 according to an embodiment. The Y-joint 7000 can be fluidly coupled and/or attached to the first and second tubes 121, 122 in the same manner as the flow splitter 110. An example of the Y-joint 7000 attached to the first and second tubes 121, 122 of the nasal cannula 10 is illustrated in FIGS. 1-6. The Y-joint 7000 can be the same as or different than the flow splitter 110. Various example dimensions and relative angles are illustrated in FIG. 7, though other dimensions and relative angles can be used in other embodiments.

The Y-joint 7000 includes a proximal tube 700 and first and second distal tubes 711, 712 that generally form a Y-shape. The proximal tube 700 extends from a proximal connector 720 to the first and second distal tubes 711, 712 along a central axis of symmetry 730. The proximal tube 700 can have a length of about 16 mm as measured along or parallel to the central axis 730. In other embodiments, the length of the proximal tube 700 can be about 14 mm to about 18 mm or any length or length range therebetween. The proximal tube 700 can have an external diameter of about 6.93 mm. In other embodiments, the external diameter of the proximal tube 700 can be about 6.5 mm to about 7.3 mm or any diameter or diameter range therebetween.

A proximal connector 720 can be attached to a proximal end 702 of the proximal tube 700. The proximal connector 720 is configured to attach to and receive gas from an air and/or oxygen source. When the Y-joint 7000 functions as the flow splitter 110, the proximal connector 720 can function as the connection end 100. The Y-joint 7000 and/or the proximal connector 720 can be rigid in some embodiments.

The proximal connector 720 can have a tapered proximal end 722 and/or a flared distal end 724 such that in cross-sectional or top view the proximal connector 720 has a trapezoidal shape (e.g., as illustrated in FIG. 7). In one example, the distal end 724 of the proximal connector 720 is about 0.8 mm wider than the outer diameter of the proximal tube 700. In other embodiments, the distal end 724 of the proximal connector 720 can be about 0.6 mm to about 1 mm wider than the outer diameter of the proximal tube 700, or any width or width range therebetween. The distal end 724 of the proximal connector 720 can have an external diameter of about 8.53 mm. In other embodiments, the external diameter of the distal end 724 of the proximal connector 720 can be about 8.2 mm to about 8.8 mm or any diameter or diameter range therebetween. The proximal end 722 of the proximal connector 720 can have the same outer diameter as the proximal tube 700.

The proximal connector 720 can have a length of about 4 mm as measured along or parallel to the central axis of symmetry 730. In other embodiments, the length of the proximal connector 720 can be about 3.5 mm to about 4.5 mm or any length or length range therebetween. The combined length of the proximal connector 720 and the proximal tube 700 can be about 20 mm. In other embodiments, the combined length of the proximal connector 720 and the proximal tube 700 can be about 16 mm to about 24 mm or any length or length range therebetween.

An inlet gas channel 740 is defined in the proximal connector 720 and the proximal tube 700 and extends therebetween along the central axis of symmetry 730. The inlet gas channel 740 can have a diameter of about 4.17 mm. In other embodiments, the diameter of the inlet gas channel 740 can be about 3.7 mm to about 4.5 mm or any diameter or diameter range therebetween. The inlet gas channel 740 corresponds to the inner diameter of the proximal connector 720 and of the proximal tube 700.

The first and second distal tubes 711, 712 extend from the proximal tube 700 along an axis 751, 752 that is disposed at an acute angle 761, 762, respectively, with respect to the central axis 730. In a preferred embodiment, each angle 761, 762 is about 45°. In other embodiments, each angle 761, 762 can be about 30° to about 60° or any angle or angle range therebetween. When the angle is about 45°, the respective axes 751, 752 of the distal tubes 711, 712 are oriented at about a 90° angle (i.e., orthogonally) with respect to each other. The distal end 781, 782 of each distal tube 711, 712 is configured to be attached (e.g., by friction, adhesive, and/or interference fit) to the first and second tubes 121, 122, respectively, of the nasal cannula 10.

Each distal tube 711, 712 has a length of about 9.08 mm as measured along the axis 751, 752 of the distal tubes 711, 712, respectively. In other embodiments, the length of each distal tube 711, 712 can be about 8.5 mm to about 9.5 mm or any length or length range therebetween. Each distal tube 711, 712 preferably has about the same length.

Each distal tube 711, 712 has an internal diameter of about 4.55 mm and external diameter of about 7.48 mm. In other embodiments, the internal diameter of each distal tube 711, 712 can be about 4.3 mm to about 4.7 mm or any diameter or diameter range therebetween. Additionally or alternatively, the external diameter of each distal tube 711, 712 can be about 7.3 mm to about 7.7 mm or any diameter or diameter range therebetween. The internal and/or external diameters of the distal tubes 711, 712 are preferably about the same.

An outlet gas channel 771, 772 is defined in each distal tube 711, 712 and extends along the axis 751, 752, respectively. The outlet gas channels 771, 772 are defined by the inner radii of the distal tubes 711, 712, respectively. As such, the outlet gas channels 771, 772 can have a diameter of about 4.3 mm to about 4.7 mm, including about 4.55 mm. The outlet gas channels 771, 772 are fluidly coupled and connected to the inlet gas channel 740 to receive the gas therefrom.

In some embodiments, a respective mechanical fillet 791, 792 can be defined between the proximal tube 700 and each distal tube 711, 712. Each mechanical fillet 791, 792 can have a fillet radius of about 0.8 mm to about 1.2 mm, including about 1 mm. Additionally or alternatively, a mechanical fillet 793 can be defined between the distal tubes 711, 712. Mechanical fillet 793 can have the same or a different fillet radius than the mechanical fillets 791, 792.

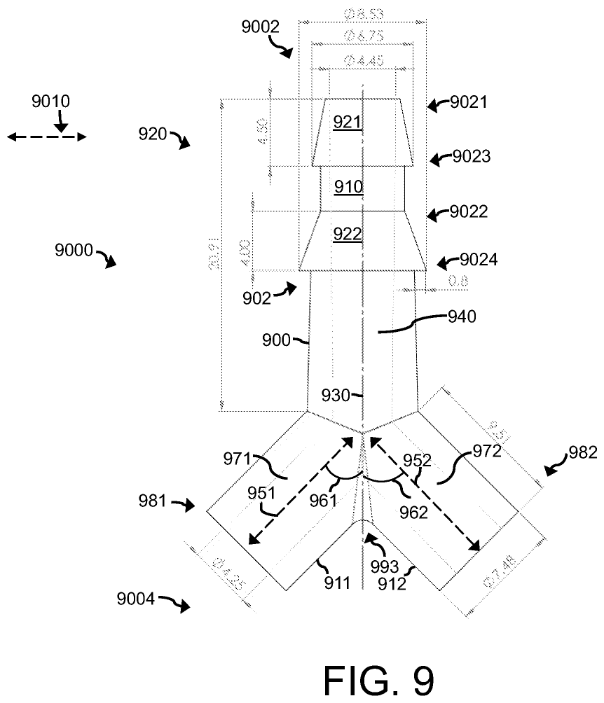

FIG. 9 is a top view of a Y-joint 9000 according to an embodiment. The Y-joint 9000 can be fluidly coupled and/or attached to the first and second tubes 121, 122 in the same manner as the flow splitter 110. An example of the Y-joint 9000 attached to the first and second tubes 121, 122 of the nasal cannula 10 is illustrated in FIG. 7. The Y-joint 9000 can be the same as or different than the flow splitter 110. Various example dimensions and relative angles are illustrated in FIG. 9, though other dimensions and relative angles can be used in other embodiments.

The Y-joint 9000 includes a proximal tube 900 and first and second distal tubes 911, 912 that generally form a Y-shape. The proximal tube 900 extends from a multi-sized proximal connector 920 to the first and second distal tubes 911, 912 along a central axis of symmetry 930. The proximal tube 900 can have a length of about 9 mm (e.g., 9.41 mm) as measured along or parallel to the central axis 930. In other embodiments, the length of the proximal tube 900 can be about 7 mm to about 11 mm or any length or length range therebetween. The proximal tube 900 can have an external diameter of about 6.95 mm. In other embodiments, the external diameter of the proximal tube 700 can be about 6.5 mm to about 7.5 mm or any diameter or diameter range therebetween.

The multi-sized proximal connector 920 can be attached to a proximal end 902 of the proximal tube 900. The multi-sized proximal connector 920 is configured to attach to and receive gas from an air and/or oxygen source. When the Y-joint 9000 functions as the flow splitter 110, the multi-sized proximal connector 920 can function as the connection end 100. The Y-joint 9000 and/or the multi-sized proximal connector 920 can be rigid in some embodiments.

The multi-sized proximal connector 920 includes first and second proximal connectors 921, 922 and an entry tube 910. The first proximal connector 921 is located at a proximal end 9002 of the Y-joint 9000. The second proximal connector 922 is located between the first proximal connector 921 and a distal end 9004 of the Y-joint 9000. For example, the second proximal connector 922 is located between the first proximal connector 921 and the proximal tube 900. The second proximal connector 922 is preferably attached to the entry tube 910 which extends from a distal end of the first proximal connector 921 to a proximal end of the second proximal connector 922. The second proximal connector 922 can be the same as proximal connector 720.

Each proximal connector 921, 922 can have a respective tapered proximal end 9021, 9022 and/or a respective flared distal end 9023, 9024 such that in cross-sectional or top view each proximal connector 921, 922 has a trapezoidal shape (e.g., as illustrated in FIG. 9). In one example, the width or external diameter of the proximal end 9021 of the first proximal connector 921, as measured with respect to an axis 9010 that is orthogonal to the central axis of symmetry 930, is about 1.75 mm narrower than the width or external diameter the distal end 9023 of the first proximal connector 921. In other embodiments, the width or external diameter of the proximal end 9021 of the first proximal connector 921 can be about 1.25 mm to about 2 mm narrower than the width or external diameter of the distal end 9023 of the first proximal connector 921, or any width or width range therebetween. In the example dimensions illustrated in FIG. 9, the width or external diameter of the proximal end 9021 of the first proximal connector 921 is about 5 mm and the width or external diameter of the distal end 9023 of the first proximal connector 921 is about 6.75 mm. In other embodiments, the width or external diameter of the proximal end 9021 of the first proximal connector 921 can be about 4.7 mm to about 5.3 mm or any width or width range therebetween, and the width or external diameter of the distal end 9023 of the first proximal connector 921 can be about 6.5 mm to about 7 mm or any width or width range therebetween.

In another example, the width or external diameter of the proximal end 9022 of the second proximal connector 922, as measured with respect to axis 9010, is about 2.9 mm narrower than the width or external diameter the distal end 9024 of the second proximal connector 922. In other embodiments, the width or external diameter of the proximal end 9022 of the second proximal connector 922 can be about 2.6 mm to about 3.2 mm narrower than the width or external diameter of the distal end 9024 of the second proximal connector 922, or any width or width range therebetween. In the example dimensions illustrated in FIG. 9, the width or external diameter of the proximal end 9022 of the second proximal connector 922 is about 5.6 mm and the width or external diameter of the distal end 9024 of the second proximal connector 922 is about 8.5 mm (e.g., 8.53 mm). In other embodiments, the width or external diameter of the proximal end 9022 of the second proximal connector 922 can be about 5.3 mm to about 5.9 mm or any width or width range therebetween, and the width or external diameter of the distal end 9024 of the second proximal connector 922 can be about 8.25 mm to about 8.75 mm or any width or width range therebetween.

The multi-sized proximal connector 920 is configured to be mechanically coupled to tubing having at least two different sizes. The first proximal connector 921 is configured to be mechanically coupled to have a tube having a relatively small internal diameter and the second proximal connector 922 is configured to be mechanically coupled to have a tube having a relatively large internal diameter.

The width or external diameter of the entry tube 910 can be the same as the width or external diameter of the proximal end 9022 of the second proximal connector 922.

The first proximal connector 921 can have a length of about 4.5 mm as measured along or parallel to the central axis of symmetry 930. In other embodiments, the length of the first proximal connector 921 can be about 4 mm to about 5 mm or any length or length range therebetween. The second proximal connector 922 can have a length of about 4 mm as measured along or parallel to the central axis of symmetry 930. In other embodiments, the length of the second proximal connector 922 can be about 3.5 mm to about 4.5 mm or any length or length range therebetween.

The combined length of the multi-sized proximal connector 920 and the proximal tube 900 can be about 21 mm (e.g., 20.91 mm) as measured along or parallel to the central axis of symmetry 930. In other embodiments, the combined length of the multi-sized proximal connector 920 and the proximal tube 900 can be about 17 mm to about 25 mm or any length or length range therebetween.

An inlet gas channel 940 is defined in the multi-sized proximal connector 920 (e.g., in the first and second proximal connectors 921, 922 and in the entry tube 910) and the proximal tube 900 and extends therebetween along the central axis of symmetry 930. The inlet gas channel 940 can have a width or diameter of about 4.5 mm (e.g., 4.45 mm). In other embodiments, the width or diameter of the inlet gas channel 940 can be about 4.3 mm to about 5.2 mm or any diameter or diameter range therebetween. The inlet gas channel 940 corresponds to the inner diameter of the first and second proximal connectors 921, 922 and of the entry tube 910.

The first and second distal tubes 911, 912 extend from the proximal tube 900 along an axis 951, 952 that is oriented at an acute angle 961, 962, respectively, with respect to the central axis 930. In a preferred embodiment, each angle 961, 962 is about 45°. In other embodiments, each angle 961, 962 can be about 30° to about 60° or any angle or angle range therebetween. When the angle is about 45°, the respective axes 951, 952 of the distal tubes 911, 912 are oriented at about a 90° angle (i.e., orthogonally) with respect to each other. The distal end 981, 982 of each distal tube 911, 912 is configured to be attached (e.g., by friction, adhesive, and/or interference fit) to the first and second tubes 121, 122, respectively, of the nasal cannula 10.

Each distal tube 911, 912 has a length of about 9.5 mm (e.g., 9.51 mm) as measured along the axis 951, 952 of the distal tube 911, 912, respectively. In other embodiments, the length of each distal tube 911, 912 can be about 8.5 mm to about 10.5 mm or any length or length range therebetween. Each distal tube 911, 912 preferably has about the same length.

Each distal tube 911, 912 has an internal diameter of about 4.25 mm and external diameter of about 7.48 mm. In other embodiments, the internal diameter of each distal tube 911, 912 can be about 4 mm to about 4.5 mm or any diameter or diameter range therebetween. Additionally or alternatively, the external diameter of each distal tube 911, 912 can be about 7.3 mm to about 7.7 mm or any diameter or diameter range therebetween. The internal and/or external diameters of the distal tubes 911, 912 are preferably about the same.

An outlet gas channel 971, 972 is defined in each distal tube 911, 912 and extends along the axis 951, 952, respectively. The outlet gas channels 971, 972 are defined by the inner radii of the distal tubes 911, 912, respectively. As such, the outlet gas channels 971, 972 can have a diameter of about 4 mm to about 4.5 mm, including about 4.25 mm. The outlet gas channels 971, 972 are fluidly coupled and connected to the inlet gas channel 940 to receive the gas therefrom.

In some embodiments, a mechanical fillet 993 can be defined between the distal tubes 911, 912. The mechanical fillet 993 can have fillet radius of about 0.8 mm to about 1.2 mm, including about 1 mm.

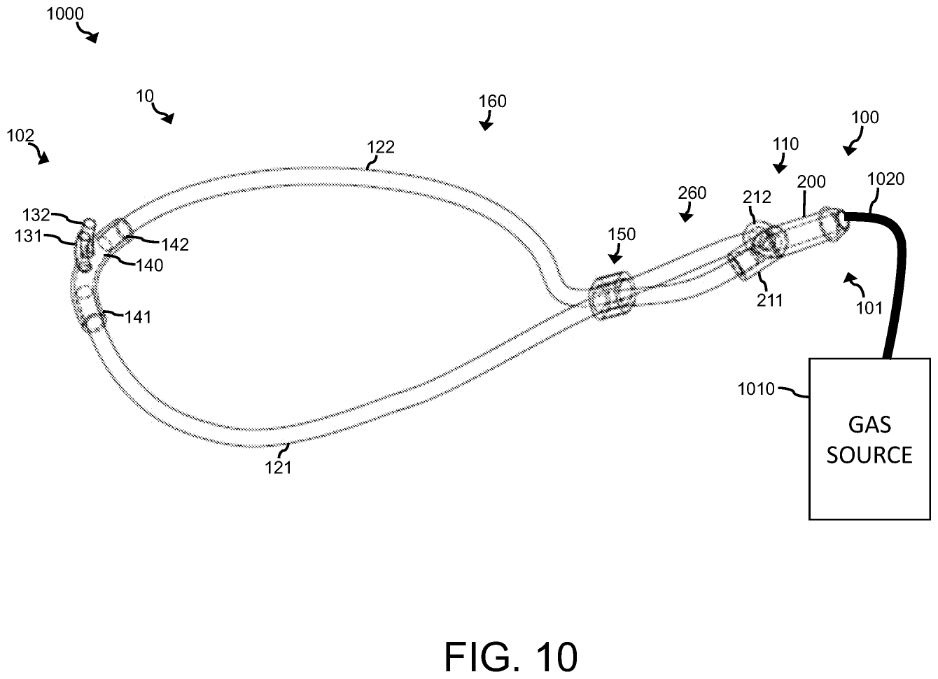

FIG. 10 is a perspective view of a system 1000 according to an embodiment. The system 1000 includes the nasal cannula 10 and a gas source 1010. The connection end 100 of the nasal cannula 10 is fluidly coupled and/or attached to an output of the gas source 1010, such as through a gas tube 1020, to receive a supply of gas from the gas source 1010. The gas supplied by the gas source 1010 can comprise or can consist of air and/or oxygen. The gas source 1010 can include a blended low-flow oxygen delivery system, such as that disclosed in U.S. Patent Application Publication No. 2018/0333555.

Those skilled in the art will understand that the present invention can be used with newborn patients as well as other patient populations such as young children generally or even adults and is not limited to a certain patient population.

The invention should not be considered limited to the particular embodiments described above, but rather should be understood to cover all aspects of the invention as fairly set out in the attached claims. Various modifications, equivalent processes, as well as numerous structures to which the invention may be applicable, will be apparent to those skilled in the art to which the invention is directed upon review of this disclosure. The claims are intended to cover such modifications and equivalents.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

1. A nasal cannula comprising:
a first tube;
a second tube;
a nasal connector tube having first and second ends, the first end connected to a distal end of the first tube, the second end connected to a distal end of the second tube;
first and second nasal prongs extending from and fluidly coupled to the nasal connector tube; and
a Y-joint having an input and first and second outputs, the first output connected to a proximal end of the first tube, the second output connected to a proximal end of the second tube.

2. The nasal cannula of claim 1, further comprising a collar having a channel through which the first and second tubes are inserted.

3. The nasal cannula of claim 2, wherein:
a loop is formed by a distal section of the first tube, a distal section of the second tube, the collar, and the nasal connector tube,
the distal section of the first tube extends from the collar to the first end of the nasal connector tube, and
the distal section of the second tube extends from the collar to the second end of the nasal connector tube.

4. The nasal cannula of claim 3, wherein the collar is configured to slidably engage the first and second tubes whereby a size of the loop is adjustable.

5. The nasal cannula of claim 3, wherein the first and second nasal prongs are curved to conform to a shape of a patient's nostrils.

6. The nasal cannula of claim 3, wherein when the first and second ends of the nasal connector tube lie approximately in a plane, the first and second nasal prongs extend from the connector tube at approximately a 45° angle with respect to the plane.

7. The nasal cannula of claim 1, wherein the Y-joint comprises:
a proximal tube;
a first distal tube; and
a second distal tube,
wherein the proximal tube is fluidly coupled to the first and second distal tubes.

8. The nasal cannula of claim 7, wherein a proximal connector is attached to a proximal end of the proximal tube.

9. The nasal cannula of claim 8, wherein the proximal connector has a flared distal end.

10. The nasal cannula of claim 9, wherein:
the first and second distal tubes extend along a respective central axis,
the Y-joint has an axis of symmetry, and
the respective central axis and the axis of symmetry form an angle having a range of about 30° to about 60°.

11. The nasal cannula of claim 10, wherein the proximal tube extends along the axis of symmetry.

12. The nasal cannula of claim 10, wherein the angle is about 45°.

13. The nasal cannula of claim 7, wherein a respective fillet is defined between (a) the first and second distal tubes and (b) the proximal tube.

14. The nasal cannula of claim 13, wherein the respective fillet has a filet radius having a range of about 0.8 mm to about 1.2 mm.

15. The nasal cannula of claim 14, wherein the filet radius is about 1 mm.

16. The nasal cannula of claim 8, wherein:
the proximal connector is a second proximal connector, and
the Y-joint further comprises a first proximal connector, the second proximal connector between the first proximal connector and the proximal tube.

17. The nasal cannula of claim 16, wherein each of the first and second proximal connectors has a flared distal end.

18. The nasal cannula of claim 16, wherein:
the first proximal connector is sized to be mechanically coupled to a first source gas tube having a first internal diameter,
the second proximal connector is sized to be mechanically coupled to a second source gas tube having a second internal diameter, and
the second internal diameter is greater than the first internal diameter.

19. A system comprising:
a gas source having a gas source output; and
a nasal cannula comprising:
a first tube;
a second tube;
a nasal connector tube having first and second ends, the first end connected to a distal end of the first tube, the second end connected to a distal end of the second tube;
first and second nasal prongs extending from and fluidly coupled to the nasal connector tube; and
a Y-joint having an input and first and second outputs, the first output connected to a proximal end of the first tube, the second output connected to a proximal end of the second tube, the input coupled to the gas source output.
